(19) United States
(12) Patent Application Publication
(10) Pub. No.: US 2026/0224808 A1
(43) Pub. Date: Aug. 6, 2026

(54) METHOD AND APPARATUS FOR CONTROLLED INFUSION VIA PULSES HAVING PREDEFINED DISCRETE BOLUS VOLUME TO ACHIEVE A CONTROLLED INFUSION RESPONSE PHARMACOKINETIC

(71) Applicant: Becton, Dickinson and Company, Franklin Lakes, NJ (US)

(72) Inventors: Steve BEGUIN, Wicklow (IE); Ludovic GIL, Grenoble, Isere (FR); Ronald PETTIS, Cary, NC (US); Natasha BOLICK, Raleigh, NC (US); Suvadra GERTH, Fuquay-Varina, NC (US)

(73) Assignee: Becton, Dickinson and Company, Franklin Lakes, NJ (US)

(21) Appl. No.: 19/159,089

(22) PCT Filed: Feb. 22, 2024
(86) PCT No.: PCT/US2024/016813

§ 371 Date: Aug. 22, 2025

Related U.S. Application Data
(60) Provisional application No. 63/447,792, filed on Feb. 23, 2023.

(51) Int. Cl. A61M 5/168; A61M 5/142

(52) U.S. Cl. CPC A61M 5/16804; A61M 5/14216; A61M 2005/14292

(57) ABSTRACT

A fluid delivery device with low mechanical compliance delivers a controlled amount of fluid via discrete fluid pulses that achieves a target drug blood concentration profile or pharmacokinetic (PK), without needing delivery to be dampened and therefore in contrast with conventional fluid delivery devices with high mechanical compliance requirements that dampen delivery to achieve a continuous delivery. The fluid delivery device operates, for example, in accordance with a pulsatile profile using an algorithm implementing a pulsatile delivery method. A pulsatile profile building method is also provided that defines the fixed interval or variable intervals in a pulsatile profile based on the PK of the drug measured (e.g., on an individual, representative model, or extracted from the PK of a population) with a single injection, and its PK is modelled using any of plural example simulation methods.

This application is a U.S. national stage of International Application No. PCT/US2024/016813, filed Feb. 22, 2024, which claims priority under 35 USC § 119 (a) from U.S. Provisional Patent Application Ser. No. 63/447,792, filed Feb. 23, 2023, the entire disclosures of each are incorporated herein by reference.

### BACKGROUND

Generally, a combination of flow resistance within a fluid path and a characteristic known as “mechanical compliance” impacts the instantaneous accuracy with which a medical fluid delivery device, such as an infusion pump, delivers an intended amount of fluid to a subject's body (e.g., a human patient). Flow resistance relates to the amount pressure required for an intended amount of fluid flow through the fluid path over a given time duration. Mechanical compliance relates to how a fluid path (i.e., that is defined by a structural body forming the path or a part of the path) expands, contracts or deflects under an environmental input, such as a pressure load from a pulse stroke from an infusion pump mechanism that is intended to deliver an amount of fluid to a catheter inserted into the patient. In addition, mechanical compliance may also comprise how a drive system deforms under load.

Existing fluid delivery devices that are designed for smooth continuous delivery of a medical fluid or medicament use a drive mechanism combined with a fluid path with high compliance. For example, an infusion pump that cooperates with an infusion set comprises high mechanical compliance, which is often used either directly or indirectly to smoothen the instantaneous flowrate at the outlet of the fluidic system. However, if an inappropriate amount of compliance exists in a fluid delivery device supposed to deliver a desired volume of fluid over a (relatively) short period of time, then said desired amount of fluid will fail to be dispensed by the device. Whereas the mechanical compliance may allow to reach smooth and controlled flowrate when backpressure and fluidic resistance as seen by the pumping mechanism remain constant, the same compliance may prevent proper control of the drug delivery flowrate when backpressure or fluidic resistance vary over time. Many of these fluid delivery devices with high compliance requirements are disadvantageous because compliance and resistance in the drive mechanism can vary over time such that delivery is not continuous and/or the intended amount of fluid is not accurately delivered to the patient. The transient accumulation of fluid in the deformed fluid path may cause the delivered volume to be below target (i.e. underdose) during a first period of time and then to be above the target (i.e. overdose) after a second period of time, thus yielding limited control of instantaneous flowrate. In the simple case of infusing drug at a constant flowrate, a variation in backpressure arising as a result of (partial) occluded fluid path or varying tissue properties of the patient may result in unwanted instantaneous flowrate variation of the drug exiting the drug delivery device and entering the target tissue. In certain cases, the underdose and overdose events may significantly affect the therapeutic outcome of the treatment.

### SUMMARY

Advantageous illustrative embodiments of the present disclosure are provided wherein a fluid delivery device with low mechanical compliance delivers a controlled amount of fluid via discrete fluid pulses that achieves a target drug blood concentration profile (i.e., a pharmacokinetic (PK)), without needing delivery to be dampened and therefore in contrast with conventional fluid delivery devices with high mechanical compliance requirements that dampen delivery to achieve a continuous delivery. The fluid delivery device operates, for example, using an algorithm implementing a pulsatile delivery method in accordance with an example embodiment.

For example, it is an aspect of illustrative embodiments to provide a method of operating an infusion pump to deliver a drug therapy fluid to a patient that comprises: generating a plurality of fluid pulses to output the fluid to a patient receiving drug therapy via the infusion pump. Each pulse among the pulses comprises a fixed volume of the fluid that is output from a fluid chamber of a pump within a time period that corresponds to a pulse duration. A number of pulses over a selected drug therapy time period and duration of an interval between successive ones of the pulses are configured in accordance with a pulsatile profile to achieve a target pharmacokinetic or PK.

In accordance with aspects of illustrative embodiments, duration of an interval is longer than the pulse duration. For example, duration of the interval is on the order of more than 10 times longer than the pulse duration.

In accordance with aspects of illustrative embodiments, the infusion pump is a rotational metering pump comprising a sleeve with an inlet port and an outlet port, and a piston that translates in the sleeve to create a fluid chamber that comprises the fixed volume after an aspirate stroke draws fluid from a reservoir into the fluid chamber via the inlet port, and the fixed volume in the fluid chamber is dispensed via the outlet port to a patient via a fluid delivery channel in fluid communication with the outlet port.

In accordance with aspects of illustrative embodiments, drug concentration of the fluid, and the fixed volume of the fluid per pulse, are used to determine at least one of the number of pulses and the duration of the interval between successive ones of the pulses.

In accordance with aspects of illustrative embodiments, the pulsatile profile comprises a plurality of pulses with corresponding intervals between successive ones of the plurality of pulses. For example, durations of the intervals are varied to achieve a target pharmacokinetic or PK.

As a further example, in accordance with aspects of illustrative embodiments, corresponding intervals between successive ones of the plurality of pulses have a maximum first interval duration, and the pulsatile profile comprises a pulse train comprising a plurality of pulses outputted with a minimal interval therebetween. The pulse train is separated from a subsequent pulse among the successive ones of the plurality of pulses by a second interval duration that is larger than the maximum first interval duration.

In accordance with illustrative embodiments of the present disclosure, an advantageous pulsatile profile building method is provided that defines the fixed interval or variable intervals in a pulsatile profile based on the pharmacokinetic of the drug measured (e.g., on an individual, representative model, or extracted from the PK of a population) with a single injection and its PK is modeled using any of plural example simulation methods.

In accordance with aspects of illustrative embodiments, the interval between the pulses in the pulsatile profile to achieve the target PK is determined by pulsatile profile building operations that comprises: using measured PK data for a single injection of the fluid; modeling the measured PK data to generate a predicted PK curve that is optimized to fit the target PK; generating respective PK traces of the predicted PK curve that correspond to different interval durations; and selecting the interval based on characteristics of the respective PK traces.

In accordance with aspects of illustrative embodiments, the modeling comprises using simulation methods chosen from a product of convolution operation and a curve-fitting operation.

In accordance with aspects of illustrative embodiments, the simulation method using a product of convolution comprises: obtaining a single dose PK curve corresponding to a unitary injection of the fluid from the measured PK data; scaling the single dose PK curve to the fixed volume of the pulse and a concentration of the fluid; superimposing the scaled single dose PK curve to the pulsatile profile using convolution; and performing a linear interpolation to the measured PK data of one of a plurality of superimposed pulse PK curves in the pulsatile profile as the predicted PK curve.

In accordance with aspects of illustrative embodiments, the simulation method using a curve-fitting operation comprises: obtaining a single dose PK curve corresponding to a unitary injection of the fluid from the measured PK data; estimating time constants from the reference PK curve; and generating the predicted PK curve using the time constants.

In accordance with aspects of illustrative embodiments, the modeling comprises: fitting the predicted PK curve to the measured PK data; varying pulse interval duration to generate the respective PK traces of the predicted PK curve; and selecting an interval based on respective characteristics of the respective PK traces.

In accordance with aspects of illustrative embodiments, one or more of the pulsatile profile building operations are iteratively performed.

In accordance with aspects of illustrative embodiments, one or more of the pulsatile profile building operations are iteratively performed to optimize the pulsatile profile.

In accordance with aspects of illustrative embodiments, the pulsatile profile building operations further comprise minimizing residuals between the modelled predicted PK and the measured PK data during the curve-fitting operation.

Additional and/or other aspects and advantages of illustrative embodiments will be set forth in the description that follows, or will be apparent from the description, or may be learned by practice of the illustrative embodiments. The illustrative embodiments may comprise apparatuses and methods for operating same having one or more of the above aspects, and/or one or more of the features and combinations thereof. The illustrative embodiments may comprise one or more of the features and/or combinations of the above aspects as recited, for example, in the attached claims.

### BRIEF DESCRIPTION OF THE DRAWINGS

The above and/or other aspects and advantages of the illustrative embodiments will be more readily appreciated from the following detailed description, taken in conjunction with the accompanying drawings, of which:

FIG. 1 is a perspective view of an example infusion pump;

FIG. 2 is a perspective view of the infusion pump in FIG. 1 with the housing cover removed to expose example pump components on a base plate;

FIGS. 3 and 4 are partial, perspective views of example pump components in an example medicament fluid delivery device that operates in accordance with a programmed pulsatile flow profile using an example pulsatile delivery method) in accordance with an illustrative embodiment of the present invention;

FIGS. 5A and 5B are perspective views of pump components of FIGS. 3 and 4 in an example medicament fluid delivery device arranged, respectively, in accordance with a ready to dispense stage of operation and a ready to aspirate stage of operation;

FIG. 5C is a perspective view of components in an example medicament fluid delivery device comprising example pump components of FIGS. 3 and 4 and associated electronic circuits on a printed circuit board;

FIG. 6A is a block diagram of components in an example medicament fluid delivery device in accordance with an illustrative embodiment of the present invention;

FIG. 6B is a schematic diagram of a medicament fluid delivery device pump motor having a current sensor in accordance with an illustrative embodiment of the present disclosure;

FIG. 7 depicts example filtered pump measurement data (e.g., motor current) from an example medicament fluid delivery device during aspirate and dispense strokes;

FIG. 8 depicts pump measurement data from an example medicament fluid delivery device indicating respective pump strokes over time and corresponding pressure changes relative the respective pump strokes;

FIG. 9 is a graph illustrating an example pharmacokinetic that varies based on and relative to a target patient's weight;

FIG. 10A is a graph illustrating example pharmacokinetics using different pulsatile profiles shown in FIGS. 10B and 10C, in accordance with an illustrative embodiment of the present disclosure;

FIG. 11A is a graph illustrating example pharmacokinetics using different pulsatile profiles shown in FIGS. 11B and 11C, in accordance with an illustrative embodiment of the present disclosure;

FIG. 12 is a graph illustrating an example pharmacokinetic of a single dose injection as example source data for a pulsatile profile building method in accordance with another embodiment of the present disclosure;

FIG. 13A is an example pulsatile profile on which a scaled PK curve is superimposed as shown in FIG. 13B for predicted blood concentrations using a pulsatile profile building method in accordance with a first example embodiment that employs a numerical simulation approach;

FIG. 14A is a graph indicating predicted blood concentrations based on different pulse intervals using a pulsatile profile building method in accordance with the first example embodiment;

FIG. 14B is a graph indicating predicted blood concentrations based on a concentration adjustment with fixed concentration and pulse interval for a designated target PK for a constant plateau and in accordance with the first example embodiment of a pulsatile profile building method;

FIG. 14C is a graph indicating predicted blood concentrations based on different animal model weights with fixed concentration and pulse interval to evaluate PK sensitivity to animal weight in accordance with the first example embodiment of a pulsatile profile building method;

FIG. 14D is a graph indicating predicted peak blood concentration versus pulse interval in accordance with the first example embodiment of a pulsatile profile building method;

FIG. 14E illustrates a predicted PK curve in accordance with the first example embodiment of a pulsatile profile building method; and

FIG. 15 illustrates a predicted PK curve using a pulsatile profile building method in accordance with a second example embodiment that employs a theoretical simulation approach.

Throughout the drawing figures, like reference numbers will be understood to refer to like elements, features and structures.

### DETAILED DESCRIPTION OF ILLUSTRATIVE EMBODIMENTS

Reference will now be made in detail to illustrative embodiments, which are depicted in the accompanying drawings. The embodiments described herein exemplify, but do not limit, the illustrative embodiments by referring to the drawings.

In accordance with example embodiments of the present disclosure, an advantageous pulsatile delivery method is described herein that achieves a target drug blood concentration profile (i.e., a pharmacokinetic (PK)) by controlling a fluid delivery device (e.g., a wearable infuser), which is configured to deliver doses of a drug therapy fluid or medicament doses using pulses of predefined, discrete, fixed bolus volume, in accordance with a designated pulsatile profile.

A pulsatile fluid flow in accordance with the present disclosure is a pulsating or intermittent dispensing of fluid using one or more of pulses, where each pulse corresponds to the delivery of a predefined, discrete, fixed fluid volume. As described below, a pulsatile profile can comprise a selected number of pulses over a selected period of time, wherein the pulses have a first interval between successive pulses that can be fixed or vary over time. The first time interval duration between successive pulses is much longer than the pulse (e.g., the time duration associated with delivery the corresponding predefined fixed bolus volume of a particular pulse). Example times associated with pulses in the illustrative pulsatile profiles provided in the accompanying drawings represent commencement of a pulse (e.g., beginning of a pulse delivery) and not necessarily the duration or time period associated with delivery of the corresponding predefined bolus. In some instances, a pulse can last a few milliseconds (ms), or up to several seconds(s). For example, discharge of the piston/sleeve chamber 38 can be on the order of a few ms. As further examples, a pulse to deliver 5 uL can last between 1-3s, and a pulse to deliver 50 uL can last approximately 250 ms. Nonetheless, as described below, the time period for delivery of the predefined bolus of a particular pulse is significantly less than the example first time interval between that pulse and an adjacent or successive pulse in a pulsatile profile. Also, as described below, a pulsatile profile can comprise one or more pulse trains, wherein a pulse train corresponds to a series of successive pulses each separated by a first time interval, followed with a pause of pulses for a second time interval that is larger than the first time interval.

In accordance with further example embodiments of the present disclosure, an advantageous pulsatile profile building method is described herein that defines the fixed interval or variable intervals in a pulsatile profile based on the pharmacokinetic (PK) of the drug measured (e.g., on an individual, representative model, or extracted from the PK of a population) with a single injection, and its PK is modeled using any of plural example simulation methods.

### Example Pulsatile Delivery Method

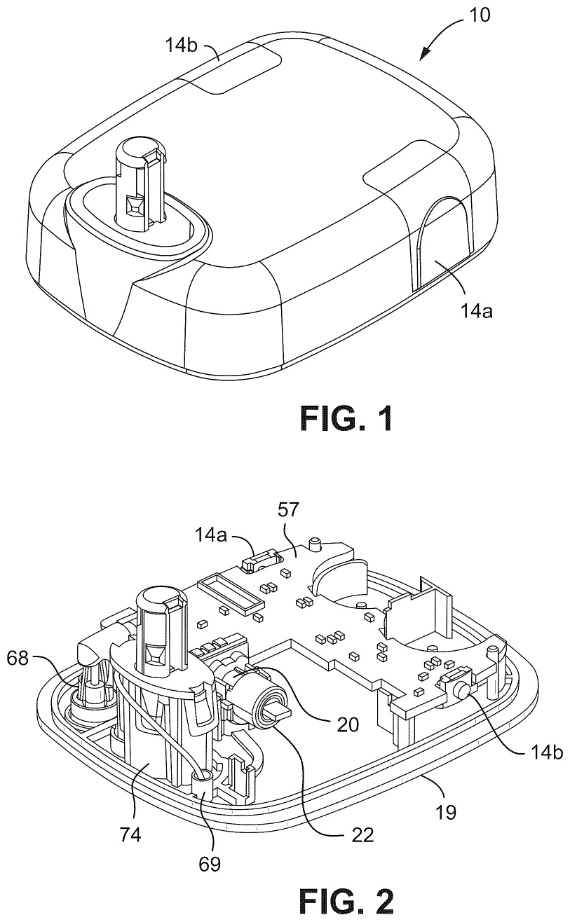

Example embodiments of a pulsatile delivery method that achieves a target PK by varying pulse interval duration using wearable drug delivery device having predefined discrete bolus volume will now be described with reference to FIGS. 1 through 11C. As described in the present disclosure, a pharmacokinetic or PK is the activity of drugs in a subject's body (e.g., a human patient) over a period of time, including the processes by which drugs are absorbed, distributed in the body, localized in the tissues, and excreted. PK can also be referred to as a target drug blood concentration profile. FIGS. 1 through 8 depict an example fluid delivery device 10 that is configured to deliver pulses of predefined discrete bolus volumes. The fluid delivery device 10 is a wearable medicament delivery device (e.g., a patch pump) with a low compliance that is advantageously configured to deliver the medicament based on a designated pulsatile profile to achieve a target PK in accordance with the pulsatile delivery method. It is to be understood that other fluid delivery devices can be controlled in accordance with the pulsatile delivery method.

FIG. 1 is a perspective view of an example fluid delivery device 10 (e.g., an infusion pump). The patch pump or wearable infusion pump 10 is described in WO 2016/048878, which is incorporated herein by reference in its entirety. The pump 10 has a housing 11, which includes a main cover 12 liquid sealed or, preferably, hermetically sealed to a base 19. The patch pump 10 can be provided with one or more user buttons indicated at 14a,14b (e.g., push button switches with associated button covers). An adhesive layer (not shown) can be provided on the base 19 for application of the infusion pump 10 to a patient's skin. The base 19 carries various components.

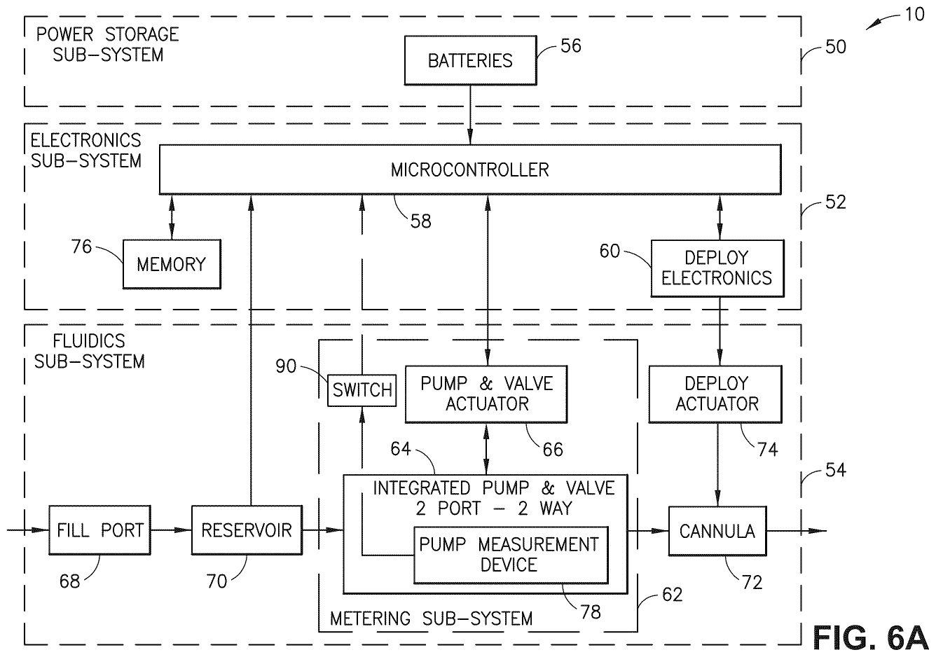

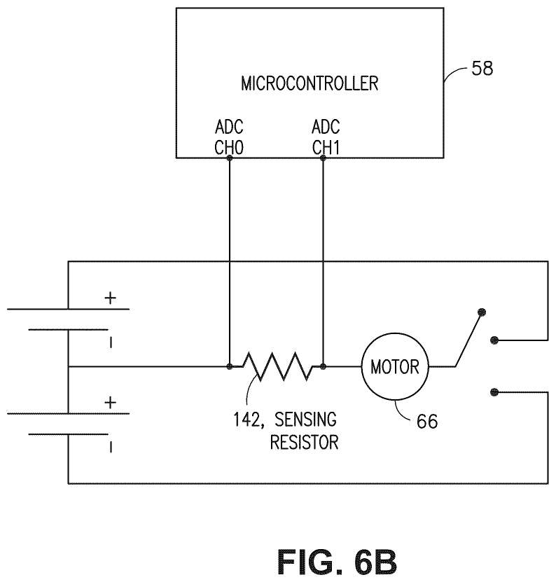

FIG. 2 illustrates some of the main components of the patch pump 10 in a perspective view with the main cover 12 and the reservoir 70 (FIG. 6A) removed for clarity. The patch pump 10 preferably includes a reservoir 70 for storing a fluid (e.g., a therapy fluid such as a medication), and a pump 64 for pumping the medication to exit the reservoir 70. The patch pump 10 also preferably includes electronics 52 for programming and operating the patch pump 10, and an insertion mechanism 74 for inserting a cannula 72 into a skin of the patient to deliver medication. According to one embodiment, a fill port 68 is a conduit for supplying a medical fluid or medicament to the reservoir 70. In some embodiments, the fill port 68 can include a portion that serves as part of the flow path for medicament exiting the reservoir 70, as described in WO 2017/053284 which is incorporated herein by reference in its entirety. A receptacle 69 is connected to the insertion mechanism 74 by tubing, for example, to transfer the medicament to the insertion mechanism 74 prior to injection into the skin of the patient.

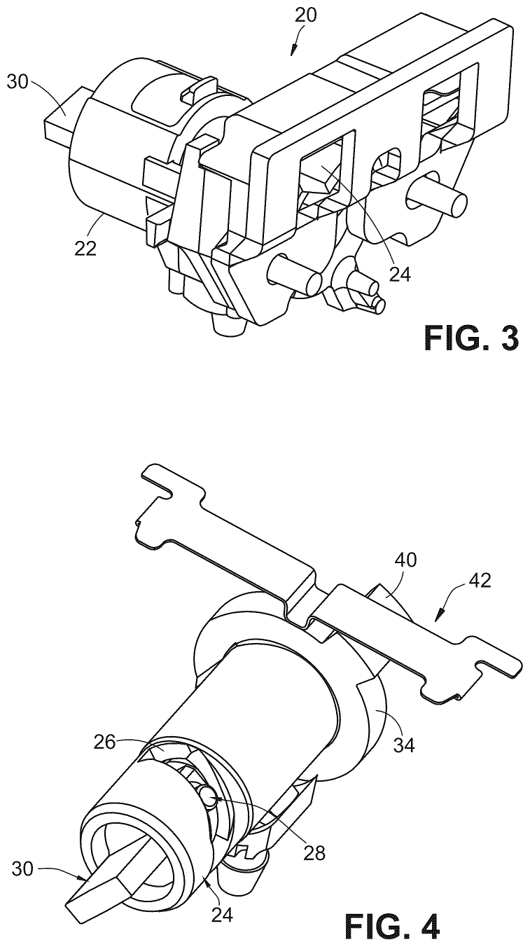

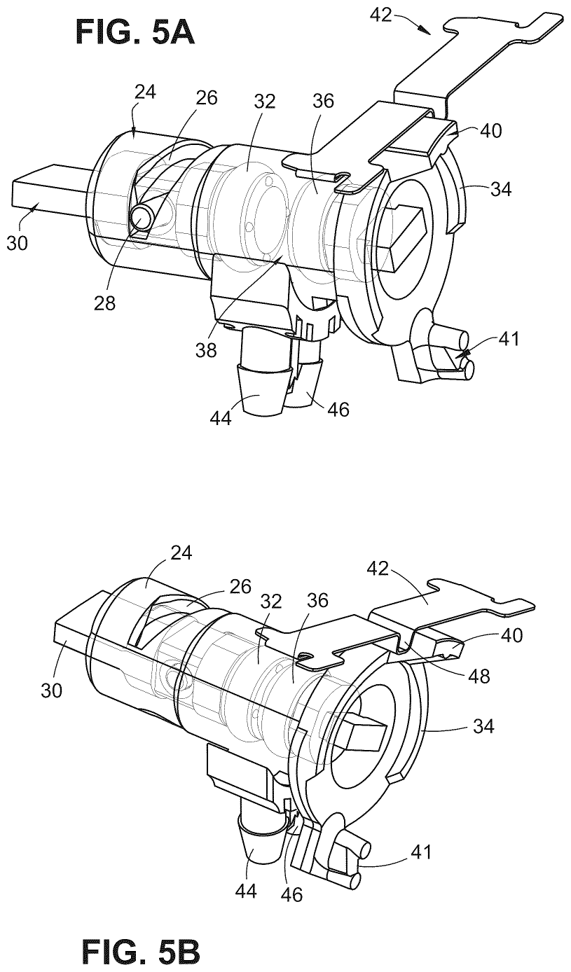

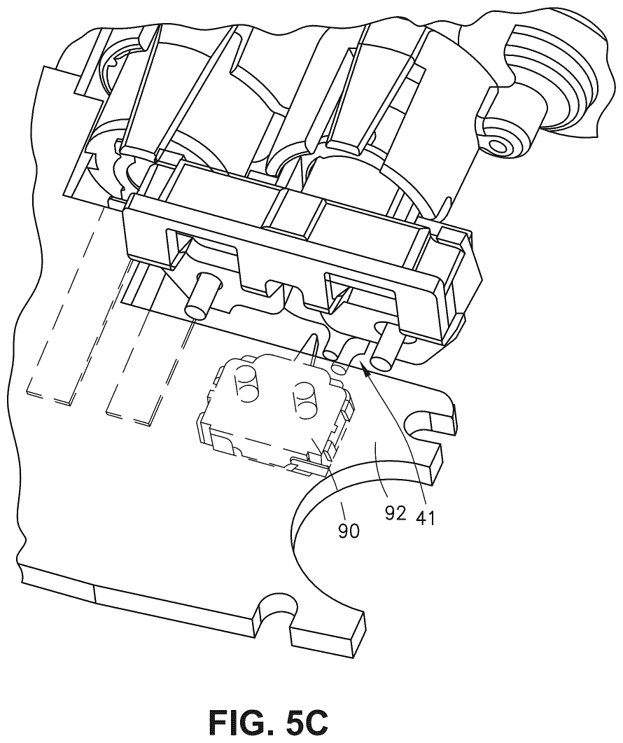

For illustrative purposes, an example infusion pump 64 will now be described with reference to FIGS. 3, 4, 5A, 5B and 5C. The pump 64 is a rotational metering-type pump described in WO 2015/157174, the content of which is incorporated herein by reference in its entirety. The pump 64 comprises a pump assembly 20 which can be connected to a DC motor and gearbox assembly (not shown) to rotate a sleeve 24 in a pump manifold 22. A helical groove 26 is provided on the sleeve. A coupling pin 28 connected to a piston 30 translates along the helical groove to guide the retraction and insertion of the piston 30 within the sleeve 24, respectively, as the sleeve 24 rotates in one direction and then rotates in the opposite direction. The sleeve has an end plug 34. Two seals 32, 36 on the respective ends of the piston and end plug that are interior to the sleeve 24 define a cavity or chamber 38 when the piston 30 is retracted, as depicted in FIG. 5A, following an aspirate stroke and therefore ready to dispense. The volume of the chamber 38 therefore changes depending on the degree of retraction of the piston 30. The volume of the chamber 38 is negligible or essentially zero when the piston 30 is fully inserted and the seals 32, 36 are substantially in contact with each other following a dispense stroke, as depicted in FIG. 5B, and therefore ready to aspirate. In accordance with an example embodiment the chamber 38 of pump 64 can be configured to correspond to the above-mentioned predefined, discrete, fixed bolus volume of a pulse. Two ports 44, 46 are provided relative to the pump manifold 22, including an inlet port 44 through which medication can flow from a reservoir 70 (FIG. 4A) for the pump 64 (FIG. 4A), and an outlet port 46 through which the medication that has been drawn into the chamber 38 (e.g., by retraction of the piston 30 during an aspirate stage of operation) can be dispensed from the chamber 38 to, for example, a fluid path to a cannula 72 (FIG. 4A) in the patient by re-insertion of the piston 30 into the chamber 38.

With continued reference to FIGS. 3, 4, 5A, 5B and 5C, the sleeve 24 can be provided with an aperture (not shown) that aligns with the outlet port 46 or the inlet port 44 (i.e., depending on the degree of rotation of the sleeve 24 and therefore the degree of translation of the piston 30) to permit the medication in the chamber 38 to flow through the corresponding one of the ports 44, 46. A pump measurement device 78 (FIG. 4A) such as a sleeve rotational limit switch can be provided which has, for example, an interlock 42 and one or more detents 40 on the sleeve 24 or its end plug 34 that cooperate with the interlock 42. The interlock 42 can be mounted to the manifold 22 at each end thereof. The detent 40 at the end face of sleeve 24 is adjacent to a bump 48 of the interlock 42 when the pump 64 is in a first position whereby a side hole in the sleeve 24 is aligned with the inlet port 44 to receive fluid from the reservoir 70 into the chamber 38. Under certain conditions, such as back pressure, it is possible that friction between the piston 30 and the sleeve 24 is sufficient to cause the sleeve 24 to rotate before the piston 30 and coupling pin 28 reach either end of the helical groove 26. This could result in an incomplete volume of liquid being pumped per dispense stroke corresponding to a pulse. In order to prevent this situation, the interlock 42 prevents the sleeve 24 from rotating until the torque passes a predetermined threshold, as shown in FIG. 5A. This ensures that piston 30 fully rotates within the sleeve until the coupling pin reaches the end of the helical groove 26. Once the coupling pin 28 hits the end of the helical groove 26, further movement by the DC motor and gearbox assembly or other type of pump and valve actuator 66 (FIG. 6A) increases torque on the sleeve 24 beyond the threshold, causing the interlock 42 to flex and permit the detent 40 to pass by the bump 48. At the completion of rotation of the sleeve 24 such that its side hole is oriented with the cannula 72 or outlet port 46, the detent 40 moves past the bump 48 in the interlock 42, as shown in FIG. 5B. Another sleeve feature 41 can be provided to engage an electrical switch (e.g., an end-stop switch 90 provided on a printed circuit board 92 and disposed relative to the sleeve and/or end plug 34 to cooperate with the pump measurement device 78 as shown in FIG. 5C)

FIG. 6A is an illustrative system diagram that illustrates example components in an example medication delivery device 10 having an infusion pump such as the pump of FIGS. 3, 4, 5A, 5B and 5C. The medicament delivery device 10 can include an electronics sub-system 52 for controlling operations of components in a fluidics sub-system 54 such as the pump 64 and an insertion mechanism 74 for deploying a cannula 72 for insertion into an infusion site on a patient's skin. A power storage sub-system 50 can include batteries 56, for example, for providing power to components in the electronics and fluidics sub-systems 52 and 54. The fluidics sub-system 54 can comprise, for example, an optional fill port 68 for filling a reservoir 70 (e.g., with medication), although the medication delivery device 10 can be optionally shipped from a manufacture having its reservoir already filled. The fluidics sub-system 54 also has a metering sub-system 62 comprising the pump 64 and a pump actuator 66. As described above, the pump 64 can have two ports 44, 46 and related valve sub-assembly that controls when fluid enters and leaves a pump chamber 38 via the respective ports 44, 46. One of the ports is an inlet port 44 through which fluid such as liquid medication flows from the reservoir 70 into the pump 64 as the result of a pump aspirate or pull stroke on a pump plunger or piston 30, for example. The other port is an outlet port 46 through which the fluid leaves the pump's chamber 38 and flows toward a cannula 72 for administration to a patient pump as the result of a pump discharge or push stroke on the pump plunger or piston 30. The pump actuator 66 can be a DC motor and gearbox assembly or other pump driving mechanism for controlling the plunger or piston 30 and other related pump parts such as a sleeve 24 that may rotate relative to the translational movement of the pump piston 30. The microcontroller 58 can be provided with an integrated or separate memory device having computer software instructions to actuate, for example, rotation of the sleeve 24 in a selected direction, translational or axial movement of a piston 30 in the sleeve 24 for an aspirate or dispense stroke, and optionally the rotation of the sleeve 24 and piston 30 together during a valve state change as described in the above-referenced WO 2015/157174. As described below, a programmed pulsatile flow profile in accordance with illustrative embodiments can be provided to the microcontroller 58 in accordance with the pulsatile delivery method described herein, to achieve a target PK. Further, the microcontroller 58 can be provided with an algorithm in accordance with a second method for pulsatile profile building as described below.

FIG. 6B shows an example apparatus for motor current sensing. A sensing resistor 142 is added to the PCB 92 to enable motor current measurement. The voltage drop on the sensing resistor 142 is provided into the analog-to-digital converter (ADC) of the microcontroller 58. The occlusion condition is then calculated by the microcontroller 58, and an occlusion or empty reservoir event is reported by the microcontroller 58 when, for example, a designated occlusion or empty reservoir motor current signature is detected. Other components can be used for current sensing to facilitate pump motor current measurement. For example, for a pulse width modulation (PWM) drive motor used as a pump actuator 66, motor current information can be extrapolated from PWM data.

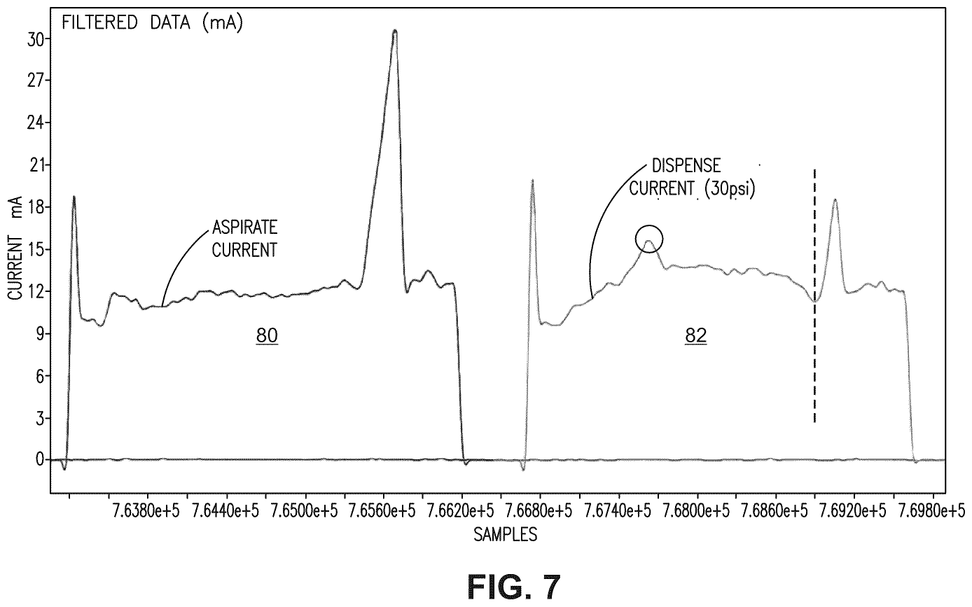

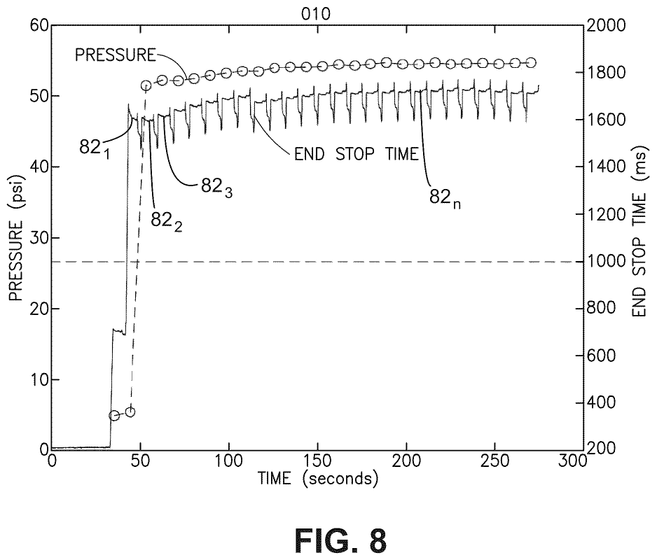

Reference is now made to FIGS. 7 and 8. FIG. 7 depicts example filtered pump measurement data (e.g., motor current) from an example delivery device 10 during an aspirate stroke 80 and a dispense stroke 82. FIG. 8 illustrates an example series of dispense strokes 82_{1 . . . n} performed by the example delivery device 10 and corresponding end stop times in an example continuous infusion operation. As stated above, one of these dispense strokes can deliver a predefined, discrete, fixed bolus volume corresponding to a pulse in accordance with example embodiments of the present disclosure. In accordance with the pulsatile delivery method described herein, an algorithm for the microcontroller 58 is configured to control the pump 64 (e.g., via a pump actuator 66 such as a motor) to deliver medicament from the reservoir 70 via the chamber 38 using pulses in a designated pulsatile profile to achieve a target PK for the medicament.

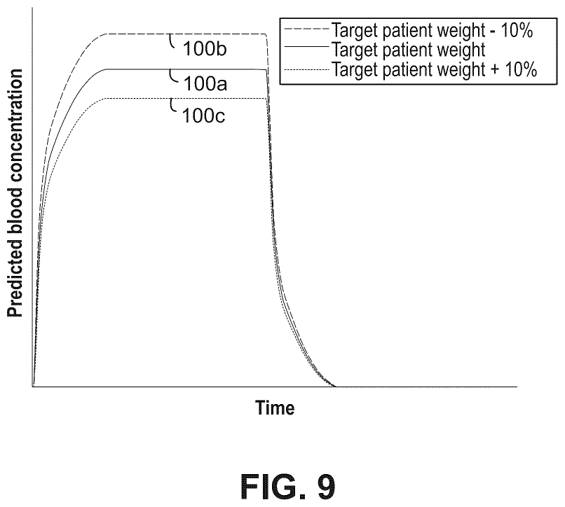

FIGS. 9 through 11C will now be referenced to describe control of a fluid delivery device to deliver pulses in accordance with an algorithm implementing the pulsatile delivery method. FIG. 9 is a graph of example curves of PKs 100 that vary based on and relative to a target patient's weight. For example, an example PK curve 100a is shown for a certain target patient weight, and higher PK curve 100b and lower PK curve 100c are shown for patient weights of −10% and +10% of the target patient weight, respectively.

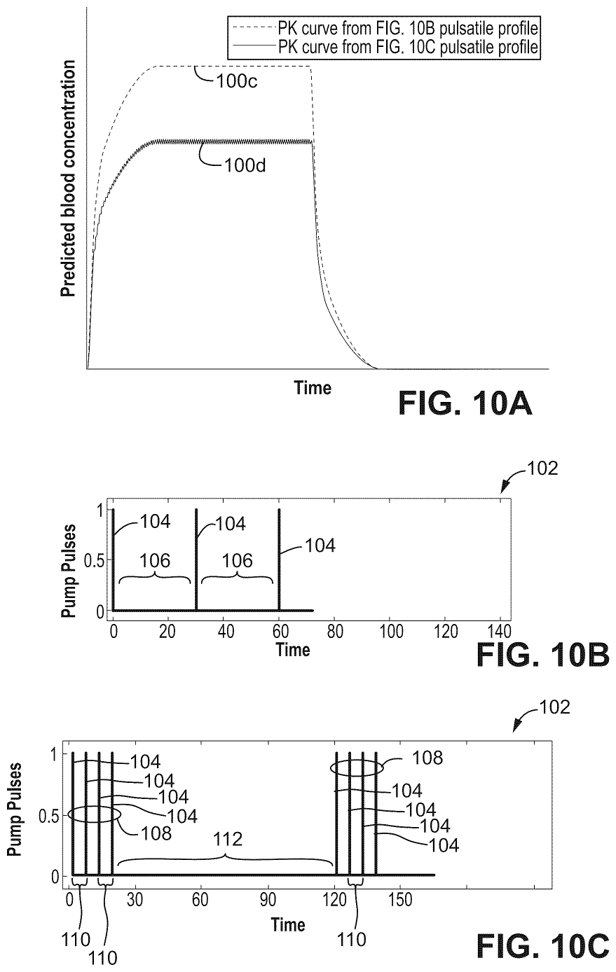

FIG. 10A is a graph of example curves of PKs 100 achieved using different pulsatile profiles 102 shown in FIGS. 10B and 10C, which illustrate impact on target PK from selected grouping and timing of pulses 104 generated via an algorithm implementing the pulsatile delivery method described herein. The higher PK curve 100c in FIG. 10A corresponds to a pulsatile profile 102 shown in FIG. 10B wherein pulses 104 are generated with intervals 106 between successive pulses. As described below, the intervals 106 need not be fixed duration but rather can vary over time and among the pulse order. The interval 106 is longer in duration than the duration of a dispense stroke operation or delivery of the fixed volume of fluid associated with the pulse (e.g., the contents of the chamber 38 in the illustrated example fluid delivery device 10) such that the pulses 104 are discrete and intermittent with respect to each other. An example range of interval 106 durations can be from milliseconds (ms) or seconds(s) to tens of minutes and possible even hours. During these intervals 106, 0 milliliters per minute (ml/min) or essentially no fluid is delivered for at least several seconds (e.g., 10 s per an example below such that the pulsatile profile 102 delivers well-controlled boluses using discrete pulses with intervals therebetween to achieve a target PK 100.

As stated above, a pulsatile fluid flow in accordance with the present disclosure is a pulsating or intermittent dispensing of fluid using one or more discrete pulses 104 where each discrete pulse corresponds to the delivery of a pre-defined fluid volume. A pulsatile profile 102 is a plurality of intermittent pulses 104, whereby each of the pulses 104 delivers a pre-defined volume of fluid with a pause or time duration 106 in between consecutive pulses that is longer than the time duration for a pulse 104 to deliver its pre-defined fluid volume via a fluid delivery device 10 controlled to deliver fluid using the pulsatile profile 102.

The lower PK curve 100d in FIG. 10A corresponds to a pulsatile profile 102 shown in FIG. 10C, and assumes a fixed injection volume and time similar to that used for the pulsatile profile 102 shown in FIG. 10B. The pulsatile profile 102 shown in FIG. 10C, however, comprises several pulses 104 delivered in a pulse train 108 and separated by a first interval of time 110, followed by a second time interval 112 that is longer than the first time interval 110 and that separates pulse trains 108. FIGS. 10A through 10C illustrate how different factors can affect a selected or designated pulsatile profile 102 chosen to achieve a target PK 100.

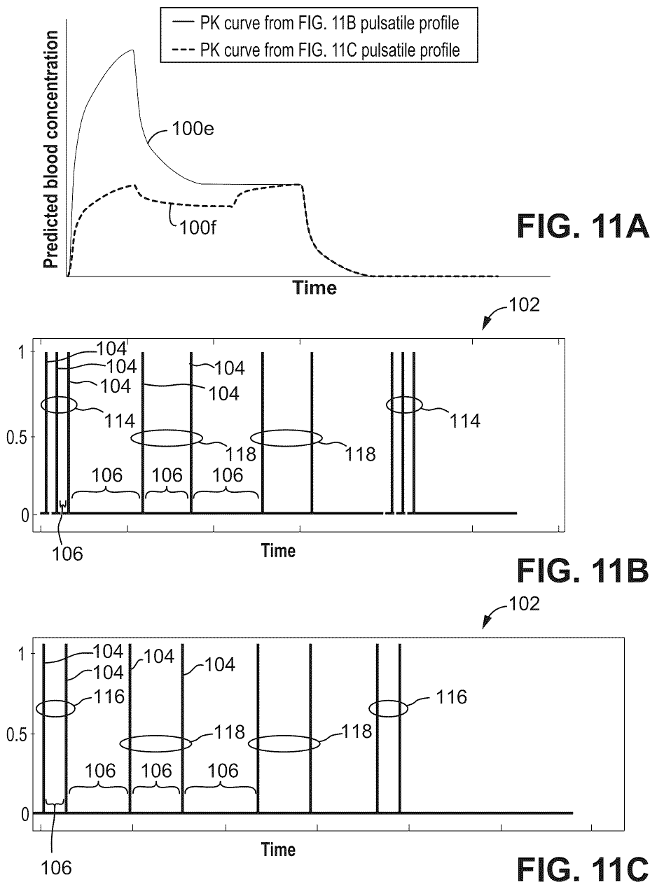

FIG. 11A is a graph of example curves of PKs 100 achieved using different pulsatile profiles 102 shown in FIGS. 11B and 11C, which illustrate impact on target PK from varying frequency of pulses 104 generated via an algorithm implementing the pulsatile delivery method described herein. The PK curve 100e in FIG. 11A corresponds to a pulsatile profile 102 shown in FIG. 11B wherein pulses 104 are generated at a relatively high frequency as indicated by 114 when compared to pulses 104 generated at a relatively low frequency as indicated by 118. The PK curve 100f in FIG. 11A corresponds to a pulsatile profile 102 shown in FIG. 11C wherein pulses 104 are generated at a relatively medium frequency as indicated by 116 when compared to pulses 104 generated at a relatively low frequency as indicated by 118 and to pulses 104 generated at a relatively high frequency as indicated by 114 in FIG. 11B.

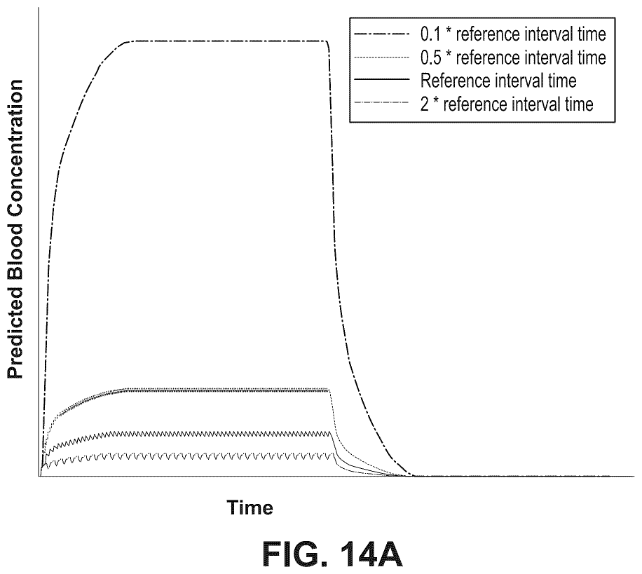

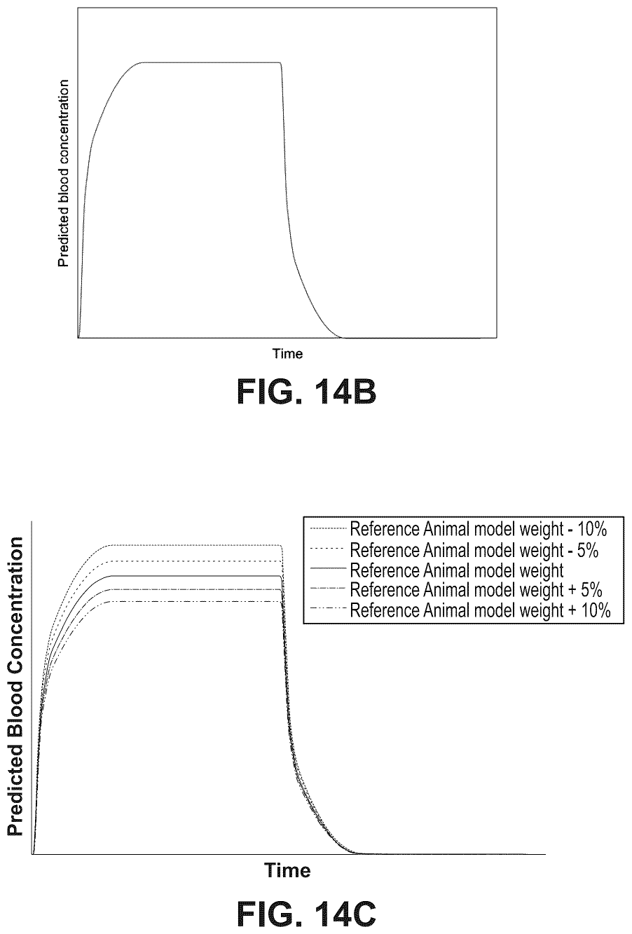

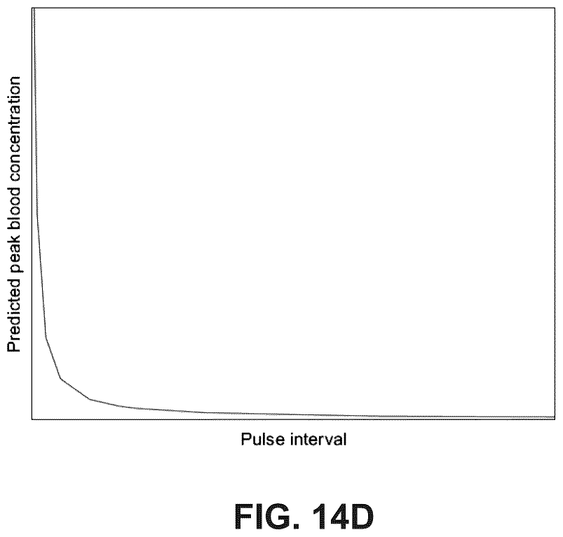

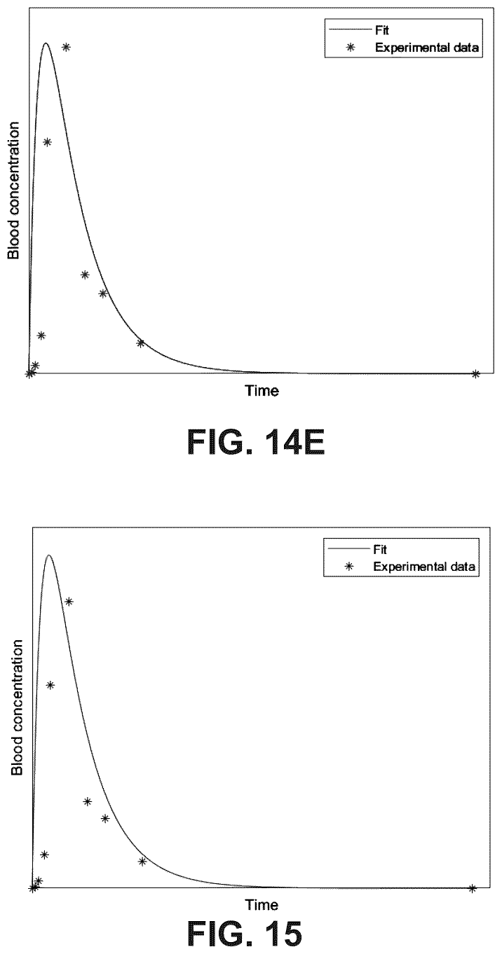

As illustrated above, a number of different embodiments for an algorithm to implement the pulsatile delivery method can be implemented, and are not limited to, the following enumerated example embodiments.
1. In accordance with an example embodiment, the interval 106 between pulses 104 is constant (e.g., as shown in the pulsatile profile 102 in FIG. 10B) and is longer (e.g., >10×) compared to the pulse (e.g., the time duration to deliver the predefined discrete bolus volume associated with the pulse). The pulses delivered by the fluid delivery device 10 under control of an algorithm implementing an embodiment of the pulsatile delivery method of the present disclosure are advantageous because they provide controlled infusion response pharmacokinetic. Such controlled discrete delivery of pulses by the fluid delivery device 10 realizes advantages over a conventional device with high compliance for continuous delivery of a medicament. If such conventional devices (e.g., a conventional syringe-style pump with leadscrew and stepper motor and change time between steps) attempted to operate in accordance with the pulsatile delivery method of the present disclosure, the intervals between steps cannot be equivalent to the discrete pulses 104 in accordance with embodiments of the present disclosure because such steps and intervals between steps are dampened by compliance within the conventional fluid delivery system. Thus, a volume delivered by such dampened steps/intervals would take a comparatively and undesirably long time compared to the shorter time during which the same volume can be delivered by the discrete bolus pulses 104 of the present disclosure.
2. In accordance with another example embodiment, the interval 106 between pulses 104 is variable (e.g., as shown in the pulsatile profile 102 in FIGS. 11B and 11C), but each interval 106 is longer (e.g., >10×) compared to the pulse 104 (e.g., the time duration to deliver the predefined discrete bolus volume associated with the pulse). Variation between pulses 104 is well-controlled, digitized and discrete in accordance with the pulsatile delivery method. An algorithm implementing the pulsatile delivery method by generating pulses 104 can change durations of intervals 106 over the course of an infusion to have impact on the PK 100. For example, an infusion may provide more frequent pulses 104 with shorter intervals 106 during certain parts of the day, or at the end versus the beginning of a treatment, and so on.
3. In a further example embodiment, a combination of successive pulses 104 and interval 106 between pulses 104 is used to achieve a desired PK. For example, a regular dose can be 5 ml per pulse 104. For a 10 ml dose, the pulsatile delivery method provides for use of two 5 ml successive pulses 104 with a long interval 106 in between the two successive pulses 104.
4. In other example embodiments, an algorithm implementing the pulsatile delivery method is configured to achieve a target PK chosen from at least one of the following types of PK curves: (1) Zero order response; (2) Steady increase; (3) Steady plateau; (4) Steady plateau of desired duration; (5) Steady decrease; (6) Cyclical pattern; (7) Cyclical pattern synchronized with diurnal/nocturnal/hormonal/biological pattern; and (8) Non-cyclical variable pattern. An example of a target PK that is a (1) Zero order response is 0 to a plateau and return to 0; that is, an increase in concentration to a plateau level as steeply or quickly as possible. An example of a target PK that is a (2) Steady increase corresponds to a blood concentration that steadily increases or ramps up over time, which can be useful to reduce side effects of a delivered medicament, for example. Alternatively, an example of a target PK that is a (5) Steady decrease corresponds to a blood concentration that steadily decreases or ramps down over time, which can be useful to reduce withdrawal effects when a patient is being taken off a certain medicament, for example. An example of a target PK that is a (3) Steady plateau is indicated by the PK curve for a 120 minute pulse interval in FIGS. 14A and 15B, which oscillates to be steady and wavy for dynamic equilibrium. This Steady plateau can be a (4) Steady plateau of desired duration, depending on the delivery instructions and efficacy of the medicament. An example of a target PK that is a (6) Cyclical pattern can be a low concentration, followed by a high concentration, followed by a low concentration, with each change in concentration occurring over a long period of time relative to the duration of treatment such as on the order of several hours over a 72 hour treatment, versus minutes or seconds. An example of a target PK that is a (7) Cyclical pattern synchronized with diurnal/nocturnal/hormonal/biological pattern can be a pulsatile profile having a pattern of delivered pulses to achieve a variable target PK over time that is synchronized with a patient's diurnal pattern (e.g., wherein the patient's activity during the day is greater than at night), or other form of chronotherapy to administer a medicament over a specific time period. As stated above, a target PK can also be a (8) Non-cyclical variable pattern.
5. In accordance with another example embodiment, the mechanical compliance of the medicament delivery device (e.g., the fluid delivery device 10 in FIGS. 1 through 5C or other fluid delivery device) is sufficiently low to allow for delivery of discrete boluses without dampening the delivery and yet still achieve continuous infusion. The fluid delivery device of the present disclosure is controlled via an algorithm, for example, using the pulsatile delivery method of the present disclosure to achieve discrete, pulsed small injections via the pulses 104 described herein, which are well-defined in time, for a target PK. By contrast, conventional fluid delivery devices have high compliance requirements and can only, through dampening, achieve constant or variable continuous flow of the fluid and do not employ pulses 104 as described in accordance with example embodiments. As stated above, if such conventional high compliance delivery devices (e.g., a conventional syringe-style pump with leadscrew and stepper motor and change time between steps) attempted to operate in accordance with the pulsatile delivery method of the present disclosure, the intervals between steps cannot be equivalent to the discrete pulses 104 in accordance with embodiments of the present disclosure because such steps and intervals between steps are dampened by compliance within the conventional fluid delivery system. Thus, a volume delivered by such dampened steps/intervals would take a comparatively and undesirably long time compared to the shorter time during which the same volume can be delivered by the discrete bolus pulses 104 of the present disclosure. Example embodiments of the present disclosure are therefore realize an advantage of providing a control window in which a known volume is injected versus continuous delivery.
6. In accordance with another example embodiment, the pulsatile profile of the medicament being delivered can drop to 0 ml/min for at least 10s between pulses 104. This embodiment illustrates a distinction from continuous infusion devices that have high compliance and require longer times than the pulsatile delivery method and related fluid delivery device to deliver the medicament.
7. In another embodiment, the pulsatile profile 102 is pre-programmed in the fluid delivery device operating in accordance with the pulsatile delivery method.
8. In yet another embodiment, one of multiple pre-programmed pulsatile profiles 1021 . . . n can be selected. For example, when one or more pre-programmed pulsatile profiles 1021 . . . n are provided to a fluid delivery device, a user can manipulate buttons or other user input devices provided on the fluid delivery device, or an a remote control or auxiliary device connected to the fluid delivery device, to select a profile 102 from among multiple stored programmed pulsatile profiles 1021 . . . n.
9. In accordance with an example embodiment, the number of successive pulses 104 in a pulsatile profile 102 can be selected to define the equivalent unitary dose. For example, the number of pulses 104, and/or the duration(s) of the interval(s) 106, 110 and/or 112, can be inputted by a user via a user interface provided on the delivery device or on a paired/connected remote control or auxiliary device. For example, input buttons 14a, 14b (FIG. 1) on the fluid delivery device 10 or a remote device can be pressed three times to deliver three doses, or can be manipulated to code number of pulses and intervals. The fluid delivery device or remote device could also have a touchscreen input or other input means.
10. In another embodiment, the interval between pulses can be selected from a pre-programmed selection in a manner described for embodiment 4(8) to select from multiple pre-programmed pulsatile profiles.
11. In an example embodiment, the number of consecutive pulses can be digitally input such as by using successive presses of a button, or otherwise programmed using an user input device or interface (e.g., a graphical user interface) on the fluid delivery device or a paired/connected remote control device or auxiliary device.
12. In an embodiment, the time interval(s) between pulses can be digitally input such as by using successive presses of a button, or otherwise programmed using an user input device or interface (e.g., a graphical user interface) on the fluid delivery device or a paired/connected remote control device or auxiliary device.

### Pulsatile Profile Building Method

In accordance with further example embodiments of the present disclosure, an advantageous pulsatile profile building method is described herein that defines the fixed interval or variable intervals in a pulsatile profile based on the pharmacokinetic (PK) of the drug measured (e.g., on an individual, representative model, or extracted from the PK of a population) with a single injection, and its PK is modeled using any of plural example simulation methods.

The pulsatile profile building method is configured to predict pharmacokinetics during pulsed infusion based on single injection PK. The pulsatile profile building method comprises simulating impact of injection parameters (e.g., one or more of pulse interval, subject weight, and drug concentration) on PK for target steady-state blood concentration. Two simulation approaches are described below; however, it is to be understood that other simulation approaches can be used. The present disclosure describes a pulsatile profile building method using a numerical approach for simulation, that is, using a product of convolution. The present disclosure also describes a pulsatile profile building method using a theoretical approach for simulation, that is, using curve fitting.

The pulsatile profile building method will now generally be described, before describing these two simulation approaches in more detail below. The pulsatile profile building method will be described with reference to a fluid delivery device such as the patch pump or infusion pump 10 described above in connection with the pulsatile delivery method. The pulsatile profile building method involves operating an infusion pump to deliver a drug therapy fluid or medicament to a patient, whereby the intervals (e.g., 106, 110 and/or 112) between the pulses 104 in the pulsatile profile 102 to achieve the target PK is determined using measured PK data for a single injection of the fluid, modeling the measured PK data to generate a predicted PK curve that is optimized to fit the target PK, generating respective PK traces of the predicted PK curve that correspond to different interval durations, and selecting an interval based on characteristics of the respective PK traces. The modeling comprises using simulation approaches chosen from a product of convolution operation and a curve-fitting operation in with example embodiments of the pulsatile profile building method.

For example, modeling by a simulation approach that uses a product of convolution can comprise:
i. obtaining a single dose PK curve corresponding to a unitary injection of the fluid from the measured PK data;
ii. scaling the single dose PK curve to the fixed volume of the pulse and a concentration of the fluid;
iii. superimposing the scaled single dose PK curve to the pulsatile profile using convolution; and
iv. performing a linear interpolation, to the measured PK data, of one of a plurality of superimposed pulse PK curves in the pulsatile profile as the predicted PK curve.

For example, modeling by a simulation approach that uses a curve-fitting operation comprises:
i. obtaining a single dose PK curve corresponding to a unitary injection of the fluid from the measured PK data;
ii. estimating time constants from a reference PK curve; and
iii. generating the predicted PK curve using the time constants.

The selecting of an interval (e.g., the intervals 106, 110 and/or 112 between the pulses 104) in a pulsatile profile 102 based on characteristics of the respective PK traces comprises, for example:
i. determining an interval by getting measured PK data for a single injection and fitting a predicted PK curve to measured PK data;
ii. varying pulse interval duration to generate respective PK traces of the predicted PK curve; and
iii. selecting an interval based on the corresponding characteristics of the respective PK traces.

### Numerical Simulation Approach Using a Product of Convolution

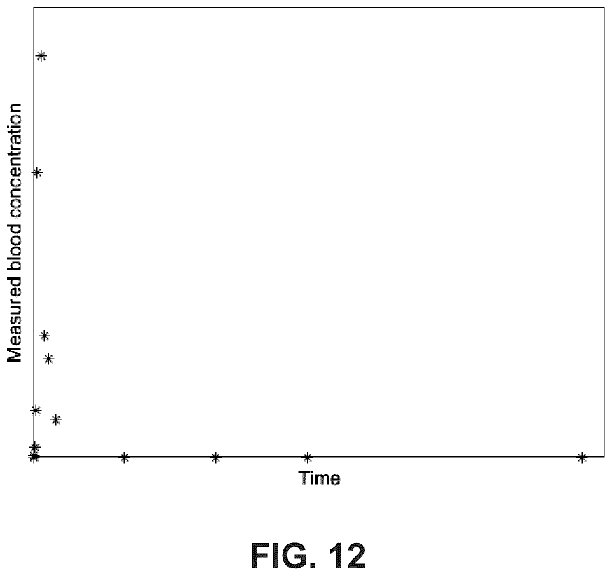

With reference to FIG. 12, source data (i.e., measured PK data) is obtained that corresponds to a reference PK curve of a single dose injection in subcutaneous (SC) tissue to obtain a simulated response as described below. In the present example, the measured PK data used for the pulsatile profile building method can be corrected for background noise (e.g., truncated and offset to 0).

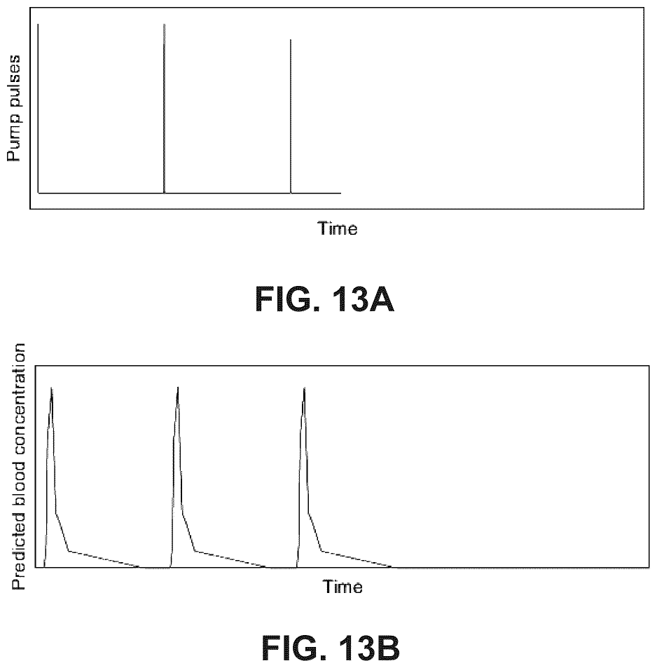

For example, the cumulative PK curve in FIG. 13B is generated in accordance with the pulsatile profile building method by scaling the single dose PK curve in FIG. 12 for a selected pulse volume and concentration. The scaled single dose PK curve is then superimposed on a pulsatile profile as shown in FIG. 13A via a product of convolution as shown in FIG. 13B.

FIG. 14A is a graph indicating predicted blood concentrations based on different pulse intervals (e.g., different values for an interval 106) with fixed concentration and subject weight. Reference to a graph like FIG. 14A for determining an interval via the pulsatile profile building method is supported by observations gained from FIGS. 14B, 14C and 14D. FIG. 14B is a graph indicating predicted blood concentrations based on a concentration adjustment with fixed concentration and pulse interval for a designated target PK that targets a constant plateau, for example. FIG. 14C is a graph indicating predicted blood concentrations based on different animal model weights (used for the single bolus PK measurement) with fixed concentration and pulse interval to evaluate PK sensitivity to animal model weight. The amplitude of the blood concentration at the plateau in the PK trace depicted in FIG. 14C is observed to be proportional to variation in subject weight (e.g., to different animal model weights as shown). FIG. 14D is a graph indicating predicted peak blood concentration versus pulse interval wherein the relationship is observed to be non-linear.

Finally, FIG. 14E illustrates a predicted PK curve in accordance with an embodiment of the pulsatile profile building method that uses a numerical simulation approach (e.g., a product of convolution). A superimposed, scaled single dose PK curve is linearly interpolated with respect to experimental data.

### Theoretical Simulation Approach Using Curve-Fitting

In the following example, the source data includes a two-species model with mass exchange characterized be the following:

$$\dot{S} = - α S - γ ( S - B )$$

$$\dot{B} = - β B + γ ( S - B ) \frac{{V}_{SubQ}}{{V}_{Blood}}$$

Where:
S is drug concentration in SubQ space
B is drug concentration in blood
α is rate of drug degradation in SubQ
β is rate of clearance in blood
γ is rate of SubQ/blood exchange

Blood volume per animal model weight is known (mL/kg). Concentration in subcutaneous (SubQ) tissue is taken at the injection point and is considered local and independent of the animal model weight. Neglecting the volume change and the local diffusion, the concentration S is equivalent to the mass of drug.

V_{SubQ} is defined as the volume of subcutaneous tissue where the mass exchange occurs and is assumed constant and equal to approximately 1 mL, for example. Dilution in blood volume was assumed to be fast due to forced convection.

In accordance with an embodiment of the pulsatile profile building method, the following three constants are estimated from a single bolus PK curve wherein animal model weight (kg) is known and the single bolus injection (mg) is known. For example:
α is about 1/(2 hr.)
β is about 1/(0.5 hr.)
γ is about 1/(1000 hr.)

The above time constants are used to generate a predicted PK curve as shown in FIG. 15. The predicted blood concentration can be analysed with respect to different parameters such as different pulse intervals (e.g., different values for an interval 106) with fixed concentration and animal model weight. For example, a graph can be generated similar to FIG. 14A, but indicating predicted blood concentrations based on different pulse intervals using a pulsatile profile building method in accordance with the second example embodiment.

Reference is now made to FIGS. 14E and 15, which depict respective predicted PK curves using a convolution-like fit and an overshoot-like fit in accordance with the first and second example embodiments of the pulsatile profile building method described above. In the example embodiments of the pulsatile profile building method described above, either simulation approach can yield a chosen interval (e.g., an x minute pulse interval 106) depending on the target PK. If, for example, a different target PK was desired such as a PK with a steady plateau, which oscillates to be steady and wavy for dynamic equilibrium (e.g., as indicated by the PK curve for a 120 minute pulse interval in FIGS. 14A and 15B), then a 2*reference interval time 106 can be chosen for a pulsatile profile. The numerical simulation approach using convolution in accordance with the first example embodiment of the pulsatile profile building method is generally considered to underestimate the plateau of the predicted PK curve (e.g., FIG. 14E), because the raw data is likely to not capture exactly the peak of blood concentration. On the other hand, the theoretical simulation approach using curve-fitting in accordance with the second example embodiment of the pulsatile profile building method is generally considered to overestimate the plateau of the predicted PK curve (e.g., FIG. 15), since the fit with raw data is generally qualitatively adjusted for meaningful good visual fit. Nonetheless, both of the simulation approaches described above for first and second example embodiments of the pulsatile profile building method yielded results with same order of magnitude within approximately 30%.

A processing device can be provided with an algorithm implementing the pulsatile profile building method. Simulation modeling can be based on source data for different medicaments besides insulin. Simulation results can be provided as an algorithm separate from and/or incorporated into a fluid delivery device for use with different medicaments and manufacturers and/or distributors thereof. For example, the algorithm of the pulsatile profile building method can be directed to various medicaments (e.g., drugs) and target concentrations that are modeled and used for different pharmaceutical companies. The algorithm of the pulsatile profile building method can configured to be iterative and to finely adjust or tune one or more parameters over time to reduce the number of iterations performed for respective medicaments (e.g., drugs) and target concentrations. As described herein, an algorithm implementing the pulsatile profile building method can use a product of convolution between a pulsed signal and a scale correction PK response of unitary single injection in accordance with an embodiment. The algorithm implementing the pulsatile profile building method can use time constants extracted from a reference PK curve injection in accordance with another embodiment. In accordance with another example embodiment, scale correction can be at least one of: reference model patient or subject weight, target patient weight, drug concentration (e.g., used in reference model single injection), drug concentration (e.g., in a fluid delivery device such as the wearable infusion pump 10), drug volume (e.g., used in reference model single injection), and bolus volume (e.g., of a fluid delivery device such as the wearable infusion pump 10). Further, in an example embodiment, the pulsatile profile 102 (e.g., number of consecutive pulses 104, and/or interval 112 between a train of pulses 108) is optimized by minimizing residuals when fitting simulated patterns on a target PK. In other words, example embodiments described herein can design an optimal pattern or pulsatile profile for injection by minimizing residuals between simulation output and target PK (e.g., the PK profile can be cyclical), and approaching to “fit curve” also defines what is optimal. Iterative processes can also be employed to achieve optimal pattern or pulsatile profile 102.

Example embodiments described herein are advantageous and realize improvements over existing medicament delivery devices because the example embodiments provide higher control of exact dose delivered using a pulsatile profile or pattern when compared to existing devices designed for continuous infusion. Further, example embodiments described herein allows for non-continuous infusion. The methods described herein (e.g., the pulsatile profile delivery method) allows use of a fluid delivery device to output discrete boluses for tunable infusion.

Existing delivery devices are disadvantageous because they are designed for smooth continuous delivery of a drug using a drive with high compliance requirements; however, compliance and resistance in their driver mechanisms vary over time such that their delivery is not continuous. Advantageous example embodiments of the present disclosure obviate need for a continuous drive and its associated complexities and its reduced performance due to inevitable variances over lifetime of continuous drive device. As demonstrated herein, using PK and pulses has demonstrable benefits over existing continuous drive mechanisms for fluid infusion. Whereas existing fluid delivery devices modulate speed of constant infusion (e.g., syringe pumps, BD Evolve™ On-body Injector, Neulasta® Onpro® on-body injector commercially available from Amgen Inc., and Omnipod® injector commercially available from Insulet Corporation), example embodiments of the present disclosure by contrast allow use of well-controlled succession of discrete injections to achieve a target PK.

It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Also, functional programs, codes, and code segments for accomplishing the illustrative embodiments can be easily construed as within the scope of claims exemplified by the illustrative embodiments by programmers skilled in the art to which the illustrative embodiments pertain. Method steps associated with the illustrative embodiments can be performed by one or more programmable processors executing a computer program, code or instructions to perform functions (e.g., by operating on input data and/or generating an output). Method steps can also be performed by, and apparatus of the illustrative embodiments can be implemented as, special purpose logic circuitry, e.g., an FPGA (field programmable gate array) or an ASIC (application-specific integrated circuit), for example.

The various illustrative logical blocks, modules, and circuits described in connection with the embodiments disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., erasable programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of claims exemplified by the illustrative embodiments. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

Computer-readable non-transitory media includes all types of computer readable media, including magnetic storage media, optical storage media, flash media and solid state storage media. It should be understood that software can be installed in and sold with a central processing unit (CPU) device. Alternatively, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. A method of operating an infusion pump to deliver a drug therapy fluid to a patient comprising:
generating a plurality of fluid pulses to output the fluid to a patient receiving drug therapy via the infusion pump;
wherein each pulse among the pulses comprises a fixed volume of the fluid that is output from a fluid chamber of a pump within a time period that corresponds to a pulse duration;
wherein a number of pulses over a selected drug therapy time period and duration of an interval between successive ones of the pulses are configured in accordance with a pulsatile profile to achieve a target pharmacokinetic or PK.

2. The method of claim 1, wherein duration of an interval is longer than the pulse duration.

3. The method of claim 2, wherein duration of the interval is on the order of more than 10 times longer than the pulse duration.

4. The method of claim 1, wherein the infusion pump is a rotational metering pump comprising a sleeve with an inlet port and an outlet port, and a piston that translates in the sleeve to create a fluid chamber that comprises the fixed volume after an aspirate stroke draws fluid from a reservoir into the fluid chamber via the inlet port, and the fixed volume in the fluid chamber is dispensed via the outlet port to a patient via a fluid delivery channel in fluid communication with the outlet port.

5. The method of claim 1, wherein drug concentration of the fluid, and the fixed volume of the fluid per pulse, are used to determine at least one of the number of pulses and the duration of the interval between successive ones of the pulses.

6. The method of claim 1, wherein the pulsatile profile comprises a plurality of pulses with corresponding intervals between successive ones of the plurality of pulses.

7. The method of claim 6, wherein the durations of the intervals are varied to achieve a target pharmacokinetic or PK.

8. The method of claim 6, wherein corresponding intervals between successive ones of the plurality of pulses have a maximum first interval duration, and wherein the pulsatile profile comprises a pulse train comprising a plurality of pulses outputted with a minimal interval therebetween, the pulse train being separated from a subsequent pulse among the successive ones of the plurality of pulses by a second interval duration that is larger than the maximum first interval duration.

9. The method of claim 1, whereby the interval between the pulses in the pulsatile profile to achieve the target PK is determined by pulsatile profile building operations comprising:
using measured PK data for a single injection of the fluid;
modeling the measured PK data to generate a predicted PK curve that is optimized to fit the target PK;
generating respective PK traces of the predicted PK curve that correspond to different interval durations; and
selecting the interval based on characteristics of the respective PK traces.

10. The method of claim 9, wherein the modeling comprises using simulation methods chosen from a product of convolution operation and a curve-fitting operation.

11. The method of claim 10, wherein the simulation method using a product of convolution comprises:
obtaining a single dose PK curve corresponding to a unitary injection of the fluid from the measured PK data;
scaling the single dose PK curve to the fixed volume of the pulse and a concentration of the fluid;
superimposing the scaled single dose PK curve to the pulsatile profile using convolution; and
performing a linear interpolation to the measured PK data of one of a plurality of superimposed pulse PK curves in the pulsatile profile as the predicted PK curve.

12. The method of claim 10, wherein the simulation method using a curve-fitting operation comprises:
obtaining a single dose PK curve corresponding to a unitary injection of the fluid from the measured PK data;
estimating time constants from the reference PK curve; and
generating the predicted PK curve using the time constants.

13. The method of claim 9, wherein the modeling comprises:
fitting the predicted PK curve to the measured PK data;
varying pulse interval duration to generate the respective PK traces of the predicted PK curve; and
selecting an interval based on respective characteristics of the respective PK traces.

14. The method of claim 9, wherein one or more of the pulsatile profile building operations are iteratively performed.

15. The method of claim 9, wherein one or more of the pulsatile profile building operations are iteratively performed to optimize the pulsatile profile.

16. The method of claim 12, wherein the pulsatile profile building operations further comprise minimizing residuals between the modelled predicted PK and the measured PK data during the curve-fitting operation.
